(19) United States
(12) Patent Application Publication
(10) Pub. No.: US 2015/0226663 A1
(43) Pub. Date: Aug. 13, 2015

(54) METHODS AND SYSTEMS FOR THE DETERMINATION OF PRESERVATIVE PENETRATION IN WOOD

(71) Applicant: Osmose Utilities Services, Inc., Buffalo, NY (US)

(72) Inventor: Jacob T. McBrayer, Locust Grove, GA (US)

(73) Assignee: Osmose Utilities Services, Inc., Buffalo, NY (US)

(21) Appl. No.: 14/175,354

(22) Filed: Feb. 7, 2014

(51) Int. Cl. G01N 21/25; G01N 33/46

(52) U.S. Cl. CPC G01N 21/251; G01N 33/46; G01N 21/255; G01N 2201/062

(57) ABSTRACT

The present invention is directed to an apparatus, system, and method for determining the extent of penetration of a preservative in wood. More particularly, the present invention is directed, in certain embodiments, to an apparatus comprising an image sensor capable of detecting a color spectrum of a sample, and a light source; a system comprising an image sensor, a light source, a sample treated with a preservative, an indicator applied to the sample, and a computing device coupled to the image sensor, wherein the computing device is capable of processing an image captured by the image sensor and determining the extent of penetration of the preservative; and methods for using such an apparatus and system to determine the extent of penetration of a preservative in wood by identifying changes in color of wood, wood products, or wood samples.

### TECHNICAL FIELD

The present invention generally pertains to systems and methods for determining

the extent, of penetration of a preservative in wood. More particularly, the present invention pertains to an apparatus comprising an image sensor capable of detecting a color spectrum of a sample, and a light source; a system comprising an image sensor, a light source, a sample treated with a preservative, an indicator applied to the sample, and a computing device coupled to the image sensor, wherein the computing device is capable of processing an image captured by the image sensor and determining the extent of penetration of the preservative; and methods for determining the extent of penetration of a preservative in wood by using an image sensor to identify changes in color of wood, wood products, or wood samples.

### BACKGROUND OF THE INVENTION

Wood can deteriorate if exposed to conditions that support growth of wood-degrading organisms. Wood and wood products are subject to attack by insects, micro-organisms, and decay fungi. These attacks may jeopardize long-term structural performance of wood and wood products. Preservatives are used to treat wood to resist insect attack and fungal decay. Preservative treatments can help greatly increase the life of wooden structures, reducing replacement costs and allowing more efficient use of forest resources. A variety of wood preservatives and application methods exist, and the degree of protection achieved may depend on the preservative used and the penetration and retention of the preservative in a wooden structure.

Preservative effectiveness is influenced not only by the protective value of the preservative chemical, but also by the method of application and extent of penetration and retention of the preservative in the treated wood. Wood preservatives include oil-type or oil-borne preservatives, waterborne preservatives that are applied as water solutions or with water as the carrier, solid particulate preservatives, fire retardants, and water repellents. Methods of application of wood preservatives include pressure processes, in which wood is impregnated in closed vessels under pressure above atmospheric pressure, and various non-pressure processes.

Penetration levels of preservatives in wood can vary widely. In most species of wood, “heartwood,” the central portion of wood that has been hardened as a result of naturally occurring chemical transformations, is more difficult to penetrate than “sapwood,” the outer portion of wood formed between the heartwood and the bark. In addition, species of wood differ in their penetrability which can affect the degree of penetration of wood preservative treatments. While slight penetration of preservative may add some value in protecting wood, deeper penetration is in many cases highly desirable.

The American Wood Protection Association (“AWPA”) provides processing and treatment requirements, including preservative penetration levels, that depend on. for example, the species, dimension, and class of wood. The AWPA also provides standard inspection methods to promote conformance in determining whether required processing and treatment requirements are met. For example, the AWPA provides standard methods for determining penetration of preservatives and fire retardants. In general, these standard methods of determining wood preservative penetration rely on the application of indicators to wood samples to facilitate visual identification of portions of wood that have been penetrated by a particular preservative.

The current invention is directed to apparatuses, systems, and methods to assist in achieving accurate, repeatable, consistent, and objective determinations of the extent of penetration of preservatives in wood and wood products.

### SUMMARY OF THE INVENTION

The present invention is directed, in certain embodiments, to an apparatus for evaluating the degree of penetration of a preservative in a sample comprising an image sensor and a light source.

In certain embodiments, the image sensor of the present invention is capable of detecting a color spectrum of the sample. In certain embodiments, the image sensor is capable of

demonstrating the degree of penetration of the preservative in the sample based on the color spectrum of the sample. In certain embodiments the image sensor comprises a high-resolution camera. In certain embodiments, an optical color filter is coupled to the image sensor.

In certain embodiments, the light source comprises a light-emitting diode. In other embodiments, the light source comprises ultraviolet light. In certain embodiments, the apparatus further comprises a sample holder. In certain embodiments, the apparatus is portable. In certain preferred embodiments, the sample is wood or a wood product.

The present invention is directed, in certain embodiments, to a system for evaluating the degree of penetration of a preservative in a sample, comprising a sample, wherein the sample has been treated with a preservative, an indicator applied to the sample, an image sensor, and a light source.

In certain embodiments, the image sensor is capable of detecting a color spectrum of a sample. In certain embodiments, the image sensor comprises a high-resolution camera. In certain embodiments, an optical color filter is coupled to the image sensor. In certain embodiments, the light source comprises a light-emitting diode. In other embodiments, the light source comprises ultraviolet light.

In certain embodiments, the system further comprises a computing device coupled to the image sensor. In certain embodiments, the computing device is capable of processing a color spectrum of the sample detected by the image sensor. In certain embodiments, the computing device is capable of determining, based on the color spectrum of the sample, a degree of penetration of a preservative in a sample. In certain embodiments, the computing device is capable of determining whether the degree of penetration of the preservative in a sample is acceptable under a pre-determined standard.

In certain embodiments, the system further comprises a sample holder. In certain embodiments, the image sensor is mounted on a stand. In certain embodiments, the image sensor and the light source are contained in a portable light-box. in certain embodiments, the portable light-box is operated in an area of minimal temperature fluctuation. In certain preferred embodiments, tire sample is wood or a wood product.

In certain embodiments, the indicator is a surrogate indicator. In certain embodiments, the preservative comprises a copper compound. In certain embodiments, the copper compound is water soluble. In certain embodiments, the copper compound is a sparingly soluble particulate. In certain embodiments, the preservative comprises a copper compound and the indicator comprises Chrome Azurol S or rubeanic acid.

The present invention is directed, in certain embodiments, to methods for evaluating the degree of penetration of a preservative in a sample comprising the steps of providing a sample, wherein the sample has been treated with a preservative, applying an indicator to the sample, and subjecting the sample to an image sensor and a light source.

In certain embodiments, the image sensor detects a color spectrum of the sample. In certain embodiments, the color spectrum of the sample is correlated with the degree of penetration of the preservative in the sample. In certain embodiments, the image sensor comprises a high-resolution camera.

In certain embodiments, the image sensor is coupled to a computing device. In

certain embodiments, the computing device processes a color spectrum of the sample detected by the image sensor. In certain embodiments, the computing device determines, based on the color spectrum of the sample, a degree of penetration of the preservative in the sample. In certain embodiments, the computing device determines whether the degree of penetration of the preservative in the sample is acceptable under a pre-determined standard.

In certain embodiments, the sample is provided in a sample holder. In certain embodiments, the image sensor and the light source are mounted on a stand. In certain embodiments, the indicator undergoes a chemical reaction with the preservative. In other embodiments, the indicator does not chemically react with the preservative. In certain preferred embodiments, the sample is wood or a wood product.

### BRIEF DESCRIPTION OF THE FIGURES

The patent or application file contains at least one drawing and photograph executed in color. Copies of this patent or patent application publication with color drawing(s) and color photograph(s) will be provided by the Office upon request and payment of the necessary fee.

Appended FIGS. 1-6 depict certain non-limiting embodiments and aspects of the present invention. The figures are not intended to limit the scope of the invention, but, instead, are intended to provide depictions of specific embodiments, features, and non-limiting characteristics of the apparatuses, systems, and methods described herein.

FIG. 1 depicts an exemplary apparatus and system of the present invention.

FIG. 2 depicts a live digital stream of wood samples in a sample holder.

FIG. 3 depicts an enhanced greyscale image of wood samples.

FIG. 4 depicts a high-resolution image of wood samples in a sample holder.

FIG. 5 depicts an enhanced color image of wood samples showing in each sample portions of heartwood, penetrated sapwood, and sapwood not penetrated by preservative.

FIG. 6 depicts a side-by-side view of an unenhanced image of wood samples and an enhanced color image of the same wood samples showing portions of heartwood, penetrated sapwood, and sapwood penetrated by preservative.

### DETAILED DESCRIPTION OF THE INVENTION

The effectiveness of any wood preservative in protecting wood, wood products, or wooden structures from degradation and decay depends upon the degree of penetration and retention of a preservative after application. As used herein, the terms “wood products,” “wood samples,” or “wooden structures” refer to any products, samples, or structures comprising wood or any other cellulosic material. Inspections of wooden structures are routinely performed to ensure compliance with quality assurance standards regarding preservative penetration and retention. The exemplary apparatuses, systems, and methods of the instant invention can assist in industry inspections by providing accurate, repeatable, and objective information on the penetration and retention of preservatives in wood.

The exemplary apparatuses, systems, and methods of the present invention may be used to determine the penetration and retention of preservatives in wood by capturing, processing and analyzing images of wood samples or wood products treated with a preservative. Images of wood samples can be acquired, processed, and analyzed with an image sensor, light source, and one or more dedicated computing devices. In exemplary embodiments of the present invention, an indicator is applied to wood treated with a preservative to produce a detectable color on the wood demonstrating the penetration and retention of the preservative. When subject to an appropriate light source, an image sensor can obtain high-resolution images of wood samples, enhance color contrasts produced by the indicator, and transmit the image to a computing device for further processing and analysis. One or more computing devices may be used to determine the extent of appropriate coloration of each wood sample, and automatically correlate the results to the penetration and retention of a wood preservative. In certain embodiments, an image may also be analyzed to determine the amount of heartwood and sapwood present in a wood product or wood sample.

Exemplary embodiments of the present invention may be used to determine the extent of penetration of preservatives in a single sample or multiple samples, depending on the desire of the user. In certain embodiments, the present invention may be used to automatically and objectively determine whether wooden structures meet industry standards with respect to wood preservative penetration and retention. In certain aspects, the present invention may be used as part of regular inspection and maintenance programs of wooden structures.

### Color Machine Vision

In its broadest sense, “color machine vision” is the use of a camera and computer together to evaluate the color of an object, similar to the way a human eye does. There are three main functions for color machine vision: “verification.” “sorting,” and “inspection.” Verification may be generally described as the use of color machine vision to ensure that a part or item matches a specified color. Sorting may be generally described as the use of color machine vision to separate parts or items based on color. Finally, inspection may be described as the use of color machine vision to find or detect defects in a part or item based on color. The instant invention applies color machine vision techniques in a novel and non-obvious way to determine the degree of penetration of wood preservatives in wood samples. Because the color a camera perceives can be changed by slight variations in temperature, controlling the temperature at which the wood samples are analyzed is an important aspect of the instant invention.

Wood Samples Treated with Preservatives

The present invention is used to evaluate the degree of penetration of a preservative in a sample. In preferred embodiments, the sample comprises wood. In certain embodiments, the wood sample comprises a portion or a cross section of a wooden structure, for example a utility pole. In alternative embodiments, the wood sample comprises an entire wood product. In preferred embodiments, the preservative comprises a wood preservative. For example, the wood preservative of the present invention may be used to prevent decay, insect attacks, and/or act as a fire retardant or water repellent. The selection of a wood preservative, or combination of wood preservatives, generally depends on the desired wood protection in the intended end use.

The invention is not limited to any specific preservative or preservative combination. In certain aspects of the present invention, a preservative applied to a wood sample or wood product comprises a waterborne preservative. Some examples of waterborne preservatives include acid copper chromate, ammoniacal copper zinc arsenate, chromated copper arsenate, alkaline copper quaternary, alkaline copper DCOI, copper bis(dimethyldithiocarbamate), copper azole, copper HDO, copper naphthenate, inorganic boron, and propiconazole and tebuconazole. Other preservatives include, but are not limited to, zinc borate, IPBC, IPBC/TEB/PPZ, oxine copper, DCOI, PTI, EL2, alkaline copper betaine (KDS), micronized copper azole, micronized copper quaternary amine, imidacloprid, and cyproconazole.

In other aspects of the present invention, a preservative applied to a wood sample

comprises a preservative in an organic-based solvent, such as oil. Examples of oil-borne or oil-type preservatives include coal-tar creosote and creosote solutions, pentachlorophenol solutions, copper naphthenate, oxine copper, zinc naphthenate, alky ammonium compounds, DCOI, and chlorpyrifos. Alternatively, a preservative may be particulate-based, for example preservative containing one or more sparingly soluble or insoluble copper-containing particulates.

Wood may be treated by immersing a wood sample in a high-pressure apparatus and applying pressure to drive the preservative into the wood. Commonly-used pressure processes of applying preservatives to a wood sample include the full cell, modified full cell, and empty cell processes. The full cell processes generally comprises sealing wood in a treating cylinder, applying a preliminary vacuum, admitting a preservative to the cylinder, applying pressure to the cylinder until the desired or maximum retention of preservative is obtained, withdrawing the preservative from the cylinder, and optionally applying a short final vacuum to free the wood from dripping preservative. The modified full cell process is similar to the full cell process except for the amount of initial vacuum and the occasional use of an extended final vacuum. Two empty-cell processes, the Rueping and the Lowry, are commonly employed and also known by those of skill in the art.

Non-pressure processes of treating wood with preservatives generally comprise surface application of preservatives by brief dipping, soaking in preservative oils or steeping in solutions of waterborne preservatives, diffusion processes with waterborne preservatives, vacuum treatment, and/or a variety of miscellaneous processes. In certain instances, preservative treatment may be applied as an in-place or remedial treatment of an in-service wooden structure.

One or more wood samples treated with one or more preservatives may be evaluated in accordance with the present invention. In certain embodiments, the wooden structure being sampled and analyzed has been previously treated with preservative. In certain embodiments, multiple samples from a single wooden structure may be taken and analyzed.

Any method known in the art of obtaining a wood sample for evaluation of

penetration of wood preservatives may be used in the present invention. In certain embodiments, an inspector may bore into a wooden structure to obtain a wood core sample. In certain embodiments, one or more samples include portions of both sapwood and heartwood of the structure. In certain embodiments, the wood sample comprises a cross-section of a wooden structure suitable for analysis in the present invention. In general, the wood samples used in accordance with the present invention should be sized such that the image sensor can capture an image of the entire sample. For instance, the wood sample may be small enough to fit within a 12 inch by 8 inch field of view.

### Penetration Indicators

An indicator may be used to evaluate the extent of penetration of a preservative in accordance with the present invention. Any indicator may be used that helps demonstrate the depth of a preservative in a wood sample by exhibiting one or more colors when subjected to a light source. In certain embodiments, an indicator may comprise more than one substance. In certain embodiments, an indicator may be applied to a wood sample after extraction of the sample from a wooden structure. The list of indicators below is exemplary and is not intended to limit the scope of indicators that may be used in the present invention.

In certain embodiments, the indicator reacts with one or more wood preservatives. For example, for boron-containing preservatives, including boron-containing fire retardants, an indicator comprising turmeric, ethyl alcohol, hydrochloric acid, and salicylic acid chemically reacts with boron to produce red colors on the wood. In other exemplary embodiments, 4, 4′*-bis-dimethylamino-triphenylmethane (DMTM) may be used to produce a green color denoting the presence of a pentachlorophenol preservative. In other exemplary embodiments, a silver-copper complex known as Penta-Check may be used to determine the presence of pentachlorophenol. In zinc-containing preservatives, an exemplary reactive indicator comprises a solution of potassium ferricyanide, potassium iodide, and starch indicator solution, which produces a deep blue color in the presence of zinc. Another exemplary preservative indicator for zinc-containing preservatives comprises dithizone (dimethylthiocarbazone) dissolved in chloroform or other chlorocarbon solvent, which produces a red color on wood if zinc is present. As another example, for phosphorus-containing fire retardants, an indicator comprising ammonium molybdate and nitric acid reacts with the preservative to produce a dark blue color in treated portions of wood.

For copper-containing wood preservatives, known reactive indicators include Chrome Azurol S (also known as Mordant Blue 29), rubeanic acid, and PAN (1-(2-pyridylazo)-2-naphthol). Chrome Azurol S chemically reacts with copper to produce a deep blue color. Chrome Azurol S has a sensitivity down to about 25 ppm copper. Rubeanic acid (dithio oxamide) solution produces a dark green color in the presence of copper, down to a sensitivity of about 25 ppm. PAN indicator reacts with copper to produces a magenta color.

A reactive indicator may be applied to a treated wood sample using any technique known in the art that permits the indicator to react with the preservative in the wood. In certain embodiments, a reactive indicator may be applied to a wood sample by spraying, dropper, or brushing. In general, time must be allowed after application of a reactive indicator to allow the indicator to adequately react with the treated wood. The appropriate amount of time for a chemical reaction to occur may vary depending on the preservative in the wood and the indicator-used. used.

One or more indicators to detect the presence of heartwood may also be applied to a wood product or wood sample using techniques known in the art, such as spraying, dropper, or brushing. An example of a heartwood indicator is o-anisidine/HCl, which produces an orange/red color in the presence of heartwood. A heartwood indicator can be used to identify the portion of heartwood in a sample. That heartwood portion can then be subtracted from the total volume of the wood sample to calculate the portion of sapwood, and ultimately the portion of sapwood penetrated by a wood preservative.

In certain aspects of the present invention, an indicator may be applied to the wood such that the indicator penetrates the wood to substantially the same extent as the preservative. These indicators, known as “surrogate indicators,” are not designed to chemically react with the preservative in the wood. In exemplary embodiments of surrogate indicators, the indicator alone can be detected to determine the extent of penetration of the preservative. Certain surrogate indicators may be specific to certain preservatives, but some preservatives may have multiple suitable surrogate indicators. In certain embodiments, surrogate indicators include ultra-violet (UV) absorbing compounds, and the presence of a surrogate indicator in wood can be evidenced by using an ultra-violet (UV) light source. The surrogate indicator may absorb light in the UV range and emit light in the visible range that is then captured by a imaging device such as, for example, a camera. Those of skill in the art will readily recognize other substances that can be used as surrogate indicators or reactive indicators to determine the penetration of preservatives in accordance with the present invention.

### Image Sensors and Light Sources

Image sensors of the present invention are capable of acquiring a high-resolution digital image of a sample. In preferred embodiments, the image sensor comprises a color high-resolution camera. In preferred embodiments, the image sensor comprises a charge-coupled device (CDD) sensor. In certain embodiments, the image sensor comprises a complementary metal oxide semiconductor (CMOS) sensor. The image sensor is generally coupled to a lens that focuses the light that contacts the image sensor. The lens may be selected to optimize the focus of the samples captured by the image sensor.

In certain embodiments, an image sensor receives light from the surface of a wood sample and converts the light into electrical signals using a charge-coupled device (CCD). In general, CCD sensors are available in cither linear or area array configurations. Linear array CCD sensors can capture a full two-dimensional image of a treated wood sample through motion of either the sample or motion of the sensor. Area array CCD sensors are capable of capturing a two-dimensional image of one or more wood samples with a single exposure to the sample. An example of a color high-resolution camera suitable for use in the present invention is the Basler scA1400-17gc.

In certain aspects of the present invention, the image sensor may be coupled to one or more optical filters that absorb certain color wavelengths, permitting only certain wavelengths to contact the sensor. The selection of an optical color filter generally depends on the color exhibited by the addition of the indicator. For instance, in embodiments of the present invention with Chrome Azurol S applied to a wood sample treated with a copper-containing preservative, an optical filter may be used that permits passage of blue color wavelengths. In certain embodiments, one or more optical filters can be fitted to a CCD camera.

A light source of the present invention illuminates the samples such that an image sensor can capture a high-resolution digital image of the samples. Illumination from the light source may be in the ultra-violet (200-400 nm). visual (400-700), or near infrared range (700-2500 nm). In preferred embodiments, the light source is a light-emitting diode (LED). As non-limiting examples, other potential light sources may include incandescent, halogen, fluorescent, and ultra-violet sources. The selection of an optimal light source and configuration can help minimize image processing procedures that occur after the image sensor has captured the images.

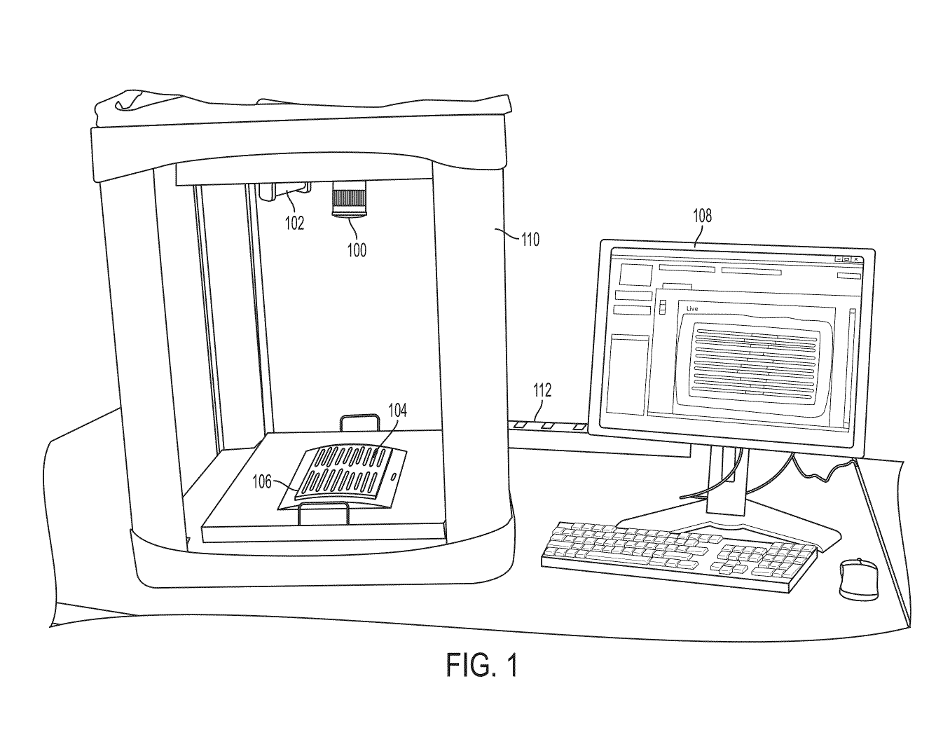

The present invention may include a light-box 110 in which the light source 102, image sensor 100, or both are mounted, as shown in FIG. 1. In certain embodiments, after obtaining one or more wood samples from a wooden structure, the wood samples 104 may be placed in a sample holder 106 within the light-box 110. In preferred embodiments, the light box 110 can reduce reflection, shadow, and some noise. In preferred embodiments, the light-box is portable. In this way, a single self-contained apparatus can be transported to the location of wood samples to be analyzed in accordance with the present invention.

In certain embodiments, the image sensor 100, the light-box 110, or both are mounted on a stand. In certain aspects, the stand may be configured such that the image sensor 100 focuses on the wood samples. In certain embodiments, the stand is foldable for ease of transport.

### Image Processing and Analysis

In certain aspects, one or more computing devices of the present invention may be coupled to the image sensor to acquire, process, and later analyze the digital image captured by the sensor. First, in certain embodiments, a computing device comprises a frame grabber that captures individual digital frames from a digital video stream. In certain embodiments, a frame grabber is coupled to a high-resolution camera. Second, a computing device may be used to perform processing operations that enhance the quality of an image. As non-limiting examples, a computing device may remove defects such as geometric distortion, improper focus, repetitive noise, inadequate or non-uniform lighting, or camera motion. In certain aspects of the present invention, a computing device then distinguishes the regions of interest of wood samples from the wood samples and produces quantitative information about the color of the wood samples, which can then he used to determine the extent of preservative penetration in the wooden structures from which the wood samples were taken.

In certain aspects of the present invention, an image captured by an image sensor

is converted into a numeric form, and pre-processed by a computing device. Pre-processing may include the initial processing of the raw image data for correction of geometric distortions, removal of noise, grey level correction, and correction for blurring, as non-limiting examples. In general, pre-processing may be designed to improve image quality by suppressing undesired distortions or the enhancement: of the treated wood samples in the image.

In certain aspects of the present invention, an image captured by an image sensor is divided into regions that have a strong correlation with the areas showing the treated wood samples. For instance, segmentation can be achieved by detection of the edge of each wood sample by edge operators, or by grouping together of similar pixels in the image, based on grey level, color, and texture, to form regions representing single objects, like a single wood sample, within the image. In certain embodiments, a computing device extracts quantitative information from segmented image regions, upon which subsequent recognition and classifications may be performed.

A computing device of the present invention may be further programmed to automatically display the extent of penetration of a preservative based on the color spectrum recognized in the image of one or more wood, samples. As an example, the computing device can be programmed to recognize the deep blue color produced by the addition of Chrome Azurol S to wood treated with copper-containing preservatives. In certain aspects, the computing device may visually display, in either color or black-and-white images, the regions of each wood sample that exhibit the required color indicating the presence of a wood preservative.

In certain aspects of the present invention, the computing device may also be pre-programmed to calculate, based on the color spectrum of a sample, the extent to which a sample has been penetrated by a preservative. For instance, a computing device may be programmed with a library of color wavelengths exhibited by the product of a selected indicator and preservative, or by an indicator when subject, to UV light. In this way, the present invention is capable of automatically providing objective and repeatable results on the extent of penetration of a preservative in multiple wood samples.

In certain embodiments, one or more computing devices of the present invention may also store in a database information obtained about a wood sample, including, but not limited to, the high-resolution images of the wood samples, the species of wood, the preservative and indicator applied, sample identification numbers, the operator performing the inspection, and any results on the extent of penetration of the samples. The computing device may also provide information on trends in wood preservative penetration, for example with respect to particular preservatives, wood species, or both.

### Methods and Systems for Evaluating the Penetration of a Preservative

In aspects of the invention, methods of the present invention are used to determine the extent of penetration of a preservative in a wooden structure. In certain embodiments, an operator may take wood samples from a wooden structure that has been treated with a preservative. In certain embodiments, a surrogate indicator has been applied to the wood sample, where the surrogate indicator is assumed to have co-migrated into the wood to substantially the same extent as the preservative. In certain embodiments, an operator applies an indicator to the wood sample, wherein the indicator is selected to chemically react with the preservative, and the product of such reaction is known to exhibit a detectable color when subject, to a light, source. If a reactive indicator is used, the operator generally waits a sufficient period of time for the indicator to chemically react with the preservative in the wood.

In certain embodiments of the present invention, the treated wood samples, to which an indicator has been applied, are placed in a sample holder 106. The wood samples are then subjected to one or more light sources and one or more image sensors. An operator may optimize the configuration of the light source and the image sensor to obtain the highest quality digital image. In certain embodiments, the wood samples 104 in the sample holder 106 may be placed in a light-box 110 to facilitate optimization of the image. The light-source 102 may be equipped with a light source 102 and an image sensor 100. In preferred embodiments, the image sensor 100 is a color high-resolution camera, and the light source 102 is an LED.

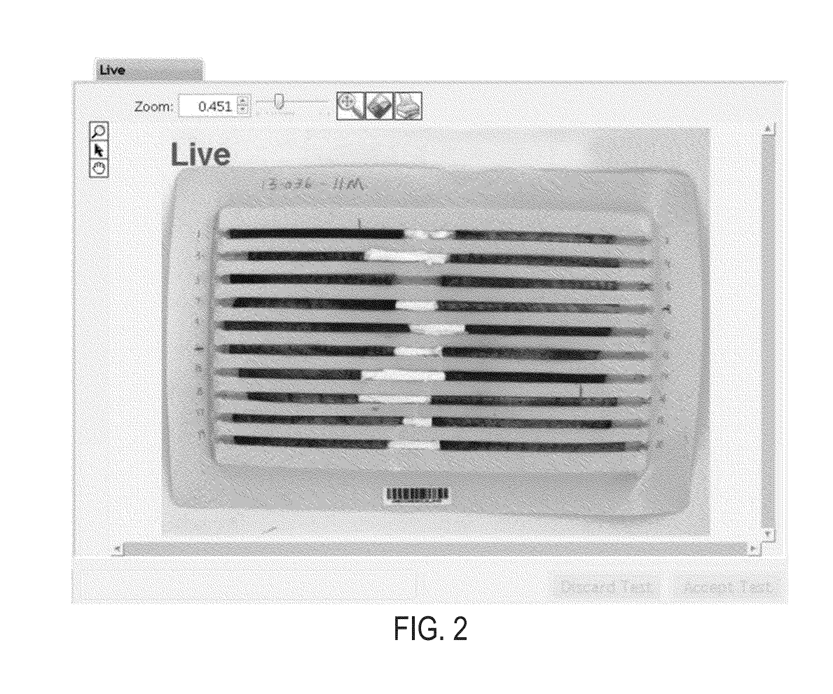

In certain preferred aspects of the present invention, an apparatus comprising a light-box 110 equipped with a light source 102 and an image sensor 100 may be coupled to a computing device 108 comprising a visual display. In preferred embodiments, the image sensor is coupled to the computing device 108 such that the image sensor 100 transmits to the computing device 108 a live digital stream of the object being targeted by the image sensor 100. The computing device 108 may then visually display the live digital stream, as shown in FIG. 2. The operator may modify the configuration of the sample holder 106, light source 102, image sensor 100, or other components based on the live digital stream to optimize the images being captured. One or more optical filters may also be added to the configuration, preferably fitted to the image sensor.

In certain aspects of the present invention, the image sensor 100 may then capture a digital image of the wood samples when subjected to the light source 102. In certain embodiments, the image sensor 100 provides a live digital stream, and a frame grabber can acquire digital still images from the stream. In certain embodiments, the frame grabber is added or built-in to the computing device 108, or, in other embodiments, the frame grabber may be built-into a high-resolution camera. The computing device 108 may then pre-process and/or enhance the digital images of the wood samples, and display the resulting high-resolution image on a visual display.

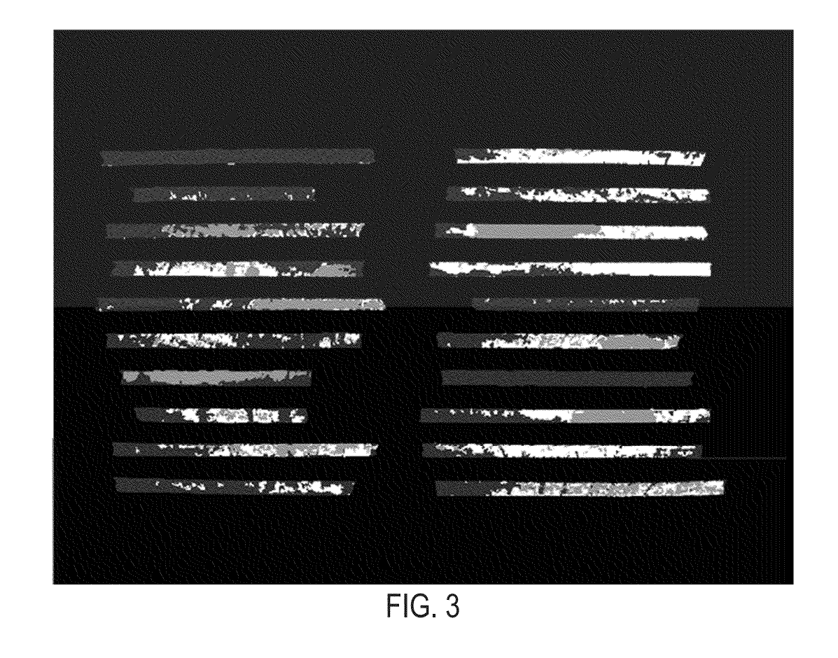

An exemplary enhanced greyscale image of wood samples treated with preservative is depicted in FIG. 3. The enhanced image of FIG. 3 depicts the same 20 wood samples shown in the live digital feed of FIG. 2. In the image of FIG. 3, the 20 wood samples were imaged and the image was enhanced to highlight the color differences shown by an indicator where a preservative had penetrated the wood. In FIG. 3, the darker regions of each sample indicate sufficient penetration of preservative, where the lightest regions of each sample show the lack of presence of the preservative.

In certain embodiments, an operator may analyze the high-resolution image

produced by the image sensor 100 to determine the extent of penetration of a wood preservative in wood samples 104. In other embodiments, an operator may analyze high-resolution images that have been processed and enhanced by a computing device 108, to determine the extent of penetration of a wood preservative in wood samples 104. In certain aspects of the present invention, because the color exhibited on the wood by the addition of an indicator to the wood samples can be captured by a digital image, which may be enhanced by optical filters or processing by a computing device, the determination of penetration of a preservative by examination of these enhanced images may be more accurate and repeatable than examination the wood sample by the human eye alone.

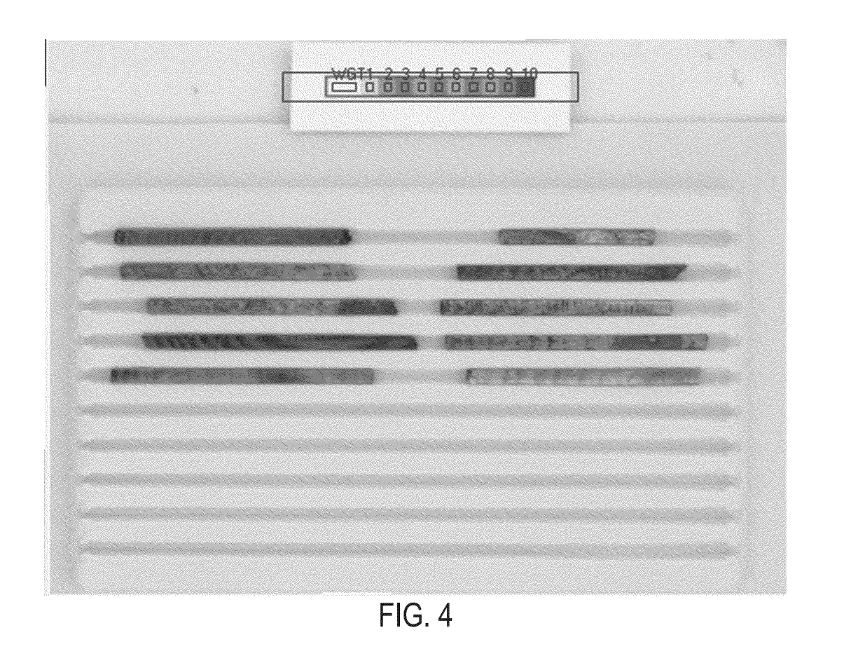

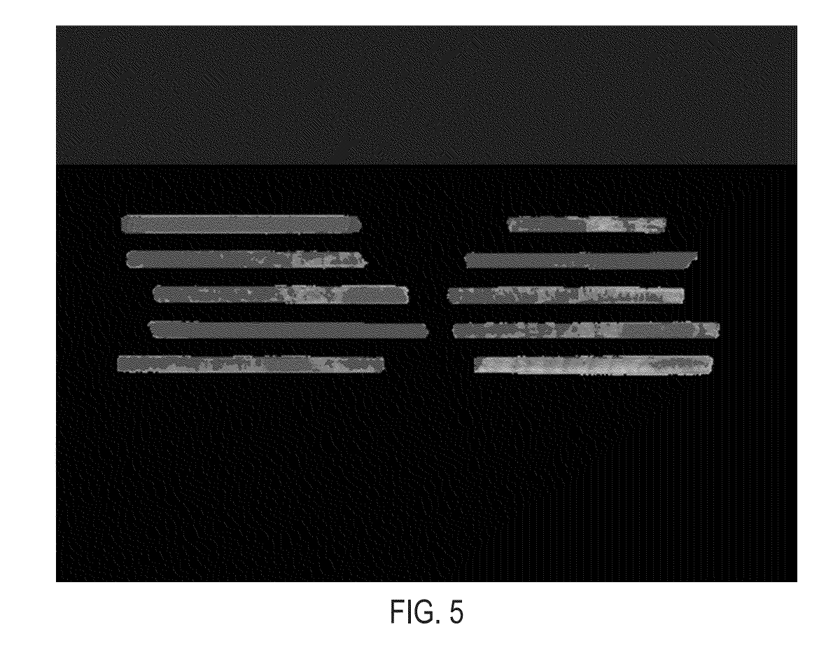

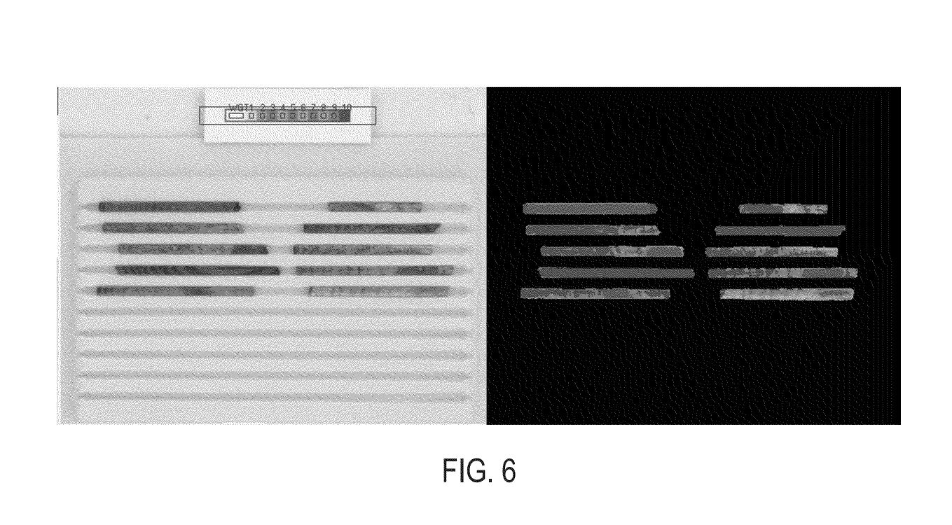

The methods of the present invention may also be used to detect the presence and percentage of heartwood, untreated sapwood, and treated sapwood in one or more wood samples. In one example, ten wood samples treated with preservatives and applied with an indicator were imaged according to methods of the present invention. The unenhanced digital image of these ten wood samples is shown in FIG. 4. The 1-10 scale at the top of FIG. 4 is used by the computing device 108 for calibration. A computing device 108 was then used to enhance the image to highlight the presence of heartwood, sapwood penetrated by preservative, and sapwood not penetrated by preservative, as shown in the image of FIG. 5. In FIG. 5, the red portions of each sample are heartwood, the blue portions are penetrated sapwood, and the remaining portions of each sample are sapwood that has not been penetrated by the preservative. Accordingly, the enhanced high-resolution image, as shown in FIG. 5, can help inspectors accurately, reproducibly and repeatedly determine penetration of preservatives in wood samples. A side-by-side comparison of the unenhanced image in FIG. 4 and the enhanced image in FIG. 5 is depicted in FIG. 6.

Exemplary methods of the present invention may also comprise further analysis

performed by a computing device 108 on the digital images captured by the image sensor 100 to objectively determine the extent of penetration of a preservative. For example, a computing device 108 coupled to the image sensor 100 may process the color spectrum of the wood samples to determine whether the extent of penetration is in compliance with one or more known pre-determined standards in the art. To reach this determination, the computing device 108 may include information about the color wavelengths exhibited by the addition to treated wood samples of various indicators known in the art. The computing device 108 may then analyze the percentage of each wood sample on a sample holder 106 that exhibits the required color (for example a deep blue produced by Chrome Azurol S in the presence of a copper-containing preservative).

In certain embodiments, the computing device 108 outputs the results of the color analysis of the wood samples as a percentage of the sample penetrated by the preservative. In certain embodiments, the computing device may calculate the percent penetrated by dividing the area of each wood sample that is sufficiently colored to indicate the presence of preservative, by the total area of each wood sample. In other embodiments, the computing device 108 automatically compares the percent penetrated to known pre-determined standards for the sampled wood species and preservatives used, to automatically determine whether the sample complies with one or more pre-determined standards of penetration provided in the art.

In certain embodiments of the present invention, the image data acquired and analyzed by the computing device 108 is logged into a central database. Each inspection performed, and the results of each inspection, may be combined with test data entered by the operator. Such test data may include, but is not limited to, information about the wood samples. the inspection, the inspector, and future maintenance or replacement steps to be taken on the wooden structure from which the samples were taken.

### EXAMPLES

The following Examples are only illustrative. It will be readily seen by one of ordinary skill in the art that the present invention fulfills all of the objectives set forth above. After reading the foregoing specification, one of ordinary skill will be able to effect various changes, substitutions of equivalents, and various other aspects of the invention as broadly disclosed therein. It. is therefore intended that the protection granted herein be limited only by the definition contained in the appended claims and equivalents thereof.

### Example 1

An exemplary system of the present invention is depicted in FIG. 1. A light source 102 and an image sensor 100 are attached inside the top of a light-box 110. The image sensor 100 in this example is a Basler scA1400-17gc 1.5 MP camera with a 16 mm lens. The light source 102 in this example is an LED. The light-box 110 is portable and has dimensions of about 3 feet (height) by about 2 feet (width) by about 2 feet (depth). The light-box 110, including the light source 102 and the image sensor 100 within the light-box, as well as the computing device 108, are connected to a power source 112. The name of the commercial exemplary embodiment of the light-box is Graftek Imaging® Lightbox PC200.

A sample holder 106 containing 20 wood samples from a wooden structure treated with a micronized copper azole preservative is placed inside the bottom of the light-box 110 as shown in FIG. 1. A rubeanic acid reactive indicator for the detection of copper-containing preservative, and an o-anisidine/HCl indictor for the detection of heartwood, have been applied to the wood samples. In this configuration, the field of view of the image sensor is 12 inches by 8 inches. The image sensor 100 is coupled to a computing device 108. A visual display of the computing device 108 provides a live digital stream of the image of the wood samples. The computing device 108 is capable of grabbing, processing, and enhancing digital still images captured by the image sensor 100, and outputting the extent of penetration of preservative in each sample based on the color spectrum exhibited by the wood samples when subjected to the LED light source.

What is claimed is:

1. An apparatus for evaluating the degree of penetration of a preservative in a sample, comprising:
an image sensor; and
a light source that illuminates a sample to provide data that may be correlated to the degree of penetration of a preservative in said sample, wherein said sample has been treated with an indicator prior to illumination by said light source.

2. The apparatus of claim 1, wherein the image sensor is capable of detecting a color spectrum of said sample.

3. The apparatus of claim 2, wherein the degree of penetration of the preservative in said sample is correlated with the color spectrum of said sample.

4. The apparatus of claim 1, wherein the image sensor comprises a high-resolution camera.

5. The apparatus of claim 1, further comprising an optical color filter coupled to said image sensor.

6. The apparatus of claim 1, wherein the light source comprises a light-emitting diode.

7. The apparatus of claim 1, wherein the light source comprises ultraviolet light.

8. The apparatus of claim 1, further comprising a sample holder.

9. The apparatus of claim 1, wherein the apparatus is portable.

10. The apparatus of claim 1, wherein said sample comprises wood or a wood product.

11. A system for evaluating the degree of penetration of a preservative in a sample, comprising:
a sample, wherein the sample has been treated with a preservative: an indicator applied to the sample;
an image sensor; and
a light source.

12. The system of claim 11, wherein the image sensor is capable of detecting a color spectrum of the sample.

13. The system of claim 11, further comprising a computing device coupled to the image sensor.

14. The system of claim 13, wherein the computing device is capable of processing a color spectrum of the sample detected by the image sensor.

15. The system of claim 14, wherein the computing device is capable of determining, based on the color spectrum of the sample, a degree of penetration of the preservative in the sample.

16. The system of claim 15, wherein the computing device is capable of determining whether the degree of penetration of the preservative in the sample is acceptable under a pre-determined standard.

17. The system of claim 11, further comprising a sample holder.

18. The system of claim 11, further comprising a stand, wherein the image sensor is mounted on a stand.

19. The system of claim 11, further comprising an optical color filter coupled to said image sensor.

20. The system of claim 11, wherein the image sensor comprises a high-resolution camera.

21. The system of claim 11, wherein the light source comprises a light-emitting diode.

22. The system of claim 11, wherein the light source comprises ultraviolet light.

23. The system of claim 21, wherein the indicator is a surrogate indicator.

24. The system of claim 11, wherein the preservative comprises a copper compound.

25. The system of claim 24, wherein the copper compound is water-soluble.

26. The system of claim 24, wherein the copper compound is an insoluble particulate.

27. The system of claim 24, wherein the indicator comprises Chrome Azurol S or rubeanic acid.

28. The system of claim 11, wherein the sample, the image sensor, and the light source are contained in a portable light-box.

29. The system of claim 28, wherein the portable light-box is operated in an area of minimal temperature fluctuation.

30. The system of claim 11, wherein said sample comprises wood or a wood product.

31. A method for evaluating the degree of penetration of a preservative in a sample, comprising the steps of:
providing a sample, wherein the sample has been treated with a preservative;
applying an indicator to the sample; and
subjecting the sample to an image sensor and a light source.

32. The method of claim 31, wherein the image sensor detects a color spectrum of the sample.

33. The method of claim 32, wherein the color spectrum of the sample is correlated with the degree of penetration of the preservative in the sample.

34. The method of claim 31, wherein the image sensor is coupled to a computing device.

35. The method of claim 34, wherein the computing device processes a color spectrum of the sample detected by the image sensor.

36. The method of claim 35, wherein the computing device determines a degree of penetration of the preservative in the sample based on the color spectrum of the sample.

37. The method of claim 36, wherein the computing device determines whether the degree of penetration of the preservative in the sample is acceptable under a pre-determined standard.

38. The method of claim 31, wherein the sample is provided in a sample holder.

39. The method of claim 38, wherein the image sensor and the light source are mounted on a stand.

40. The method of claim 31, wherein the image sensor comprises a high-resolution camera.

41. The method of claim 31, wherein said indicator undergoes a chemical reaction with said preservative.

42. The method of claim 41, wherein a product of said chemical reaction has a detectable color.

43. The method of claim 31, wherein said indicator does not chemically react with said preservative.

44. The method of claim 31, wherein said sample comprises wood or a wood product.
